(19) United States
(12) Patent Application Publication
(10) Pub. No.: US 2023/0211128 A1
(43) Pub. Date: Jul. 6, 2023

(54) Dressing-Based Traction Apparatus with Integrated Tourniquet for Improved Blood Draw

(71) Applicant: Becton, Dickinson and Company, Franklin Lakes, NJ (US)

(72) Inventors: Jonathan Karl Burkholz, Salt Lake City, UT (US); Megan S. Scherich, Salt Lake City, UT (US); Yiping Ma, Layton, UT (US)

(21) Appl. No.: 18/093,176

(22) Filed: Jan. 4, 2023

Related U.S. Application Data
(60) Provisional application No. 63/296,702, filed on Jan. 5, 2022.

(51) Int. Cl. A61M 25/02

(52) U.S. Cl. CPC A61M 25/02; A61M 2025/0266

(57) ABSTRACT

Provided herein is a catheter assembly, including a catheter adapter having a proximal end, a distal end, and a lumen extending along a first longitudinal axis therebetween, a catheter extending from the distal end of the catheter adapter, a dressing comprising a proximal end, a distal end, and first and second sides extending therebetween, the dressing defining a second longitudinal axis and configured to cover at least a portion of the catheter adapter at a site of penetration into a patient's vasculature, the dressing further comprising an upper surface and a lower surface, the lower surface configured to contact skin of a patient, and a tourniquet configured to apply a radially-inward pressure to a limb of a patient near the site of penetration, the tourniquet configured to couple with the dressing.

### CROSS-REFERENCE TO RELATED APPLICATION

The present application claims priority to U.S. Provisional Application No. 63/296,702 entitled “Dressing-Based Traction Apparatus with Integrated Tourniquet for Improved Blood Draw” filed Jan. 5, 2022, the entire disclosure of which is hereby incorporated by reference.

### BACKGROUND OF THE INVENTION

### Field of the Invention

The present disclosure relates generally to intravenous (IV) catheter assemblies and, more specifically, to IV catheter assemblies with traction devices and tourniquets for improving blood collection through indwelling catheters.

### Description of Related Art

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of IV catheter device includes a catheter that is over-the-needle. As its name implies, the catheter that is over-the-needle may be mounted over an introducer needle having a sharp distal tip. The IV catheter device may include a catheter adapter, the catheter extending distally from the catheter adapter, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient, and the catheter is left in place as an indwelling catheter.

Over time, indwelling IV catheter devices can become occluded at or near the tip of the catheter due to the presence of a fibrin sheath, thrombus, vein walls, or valves. Occlusions can limit the functionality of the catheter for infusion and/or aspiration, including the successful acquisition of blood from a patient. There are times when current catheters are manipulated manually to enable blood draw; however, doing so in an uncontrolled way, can increase incidence of dislodgement, shorten the life of the catheter indwell. Thus, a need exists in the art for improved devices, systems, and methods of manipulating an indwelling catheter to free the catheter tip and allow for blood collection and fluid delivery therefrom or therethrough.

### SUMMARY OF THE INVENTION

Provided herein is a catheter assembly, including a catheter adapter having a proximal end, a distal end, and a lumen extending along a first longitudinal axis therebetween, a catheter extending from the distal end of the catheter adapter, a dressing having a first end, a second end, and first and second sides extending therebetween, the dressing defining a second longitudinal axis and configured to cover at least a portion of the catheter adapter at a site of penetration into a patient's vasculature, the dressing further having an upper surface and a lower surface, the lower surface configured to contact skin of a patient, and a tourniquet configured to apply a radially-inward pressure to a limb of a patient near the site of penetration, the tourniquet configured to couple with the dressing.

In accordance with an embodiment of the present invention, the tourniquet includes one or more arms configured to couple with the dressing.

In accordance with an embodiment of the present invention, the one or more arms are configured to be coupled with the dressing at one or more locations along the arms.

In accordance with an embodiment of the present invention, the dressing further includes one or more pegs arranged on the upper surface.

In accordance with an embodiment of the present invention, the one or more pegs are arranged along the second longitudinal axis of the dressing, the second longitudinal axis being parallel to the first longitudinal axis.

In accordance with an embodiment of the present invention, the one or more pegs includes at least one peg arranged along the first side and at least one peg arranged along the second side.

In accordance with an embodiment of the present invention, the one or more arms are configured to releasably couple to the one or more pegs.

In accordance with an embodiment of the present invention, the one or more arms include a plurality of openings, the openings configured to releasably couple to the one or more pegs.

In accordance with an embodiment of the present invention, the one or more arms are connected to the tourniquet at a plurality of locations.

In accordance with an embodiment of the present invention, the one or more arms are coupled with the dressing at a single location.

In accordance with an embodiment of the present invention, the one or more arms include a tensioning assembly.

In accordance with an embodiment of the present invention, the one or more arms couple to the dressing adjacent the proximal end of the catheter adapter.

In accordance with an embodiment of the present invention, the one or more arms couple to the dressing adjacent the distal end of the catheter adapter.

In accordance with an embodiment of the present invention, the one or more arms are formed of a flexible material.

In accordance with an embodiment of the present invention, the one or more arms are formed of an elastomeric material.

In accordance with an embodiment of the present invention, the one or more arms are formed of a rigid material.

In accordance with an embodiment of the present invention, the dressing includes an adhesive arranged on the lower surface.

In accordance with an embodiment of the present invention, an enclosure is configured to cover at least a portion of the catheter adapter.

In accordance with an embodiment of the present invention, the tourniquet includes a fastener configured to allow the tourniquet to be shortened and lengthened, thereby allowing differing amounts of radially-inward pressure to be applied to the patient's limb.

In accordance with an embodiment of the present invention, a second tourniquet is configured to apply a radially-inward pressure to a limb of a patient near the site of penetration, the second tourniquet configured to couple with the dressing.

In accordance with an embodiment of the present invention, the second tourniquet includes one or more second arms configured to couple with the dressing.

Also provided herein is a catheter assembly, including a catheter adapter comprising a proximal end, a distal end, and a lumen extending along a first longitudinal axis therebetween, a catheter extending from the distal end of the catheter adapter, a tourniquet configured to apply a radially-inward pressure to a limb of a patient near the site of penetration, the tourniquet configured to be coupled with the catheter adapter.

In accordance with an embodiment of the present invention, the tourniquet includes one or more first arms.

In accordance with an embodiment of the present invention, the catheter adapter includes a pair of wings arranged about the lumen.

In accordance with an embodiment of the present invention, the catheter adapted further includes one or more pegs arranged on an upper surface thereof.

In accordance with an embodiment of the present invention, the one or more pegs are arranged on the pair of wings.

In accordance with an embodiment of the present invention, the one or more first arms are configured to releasably couple to the one or more pegs.

In accordance with an embodiment of the present invention, the one or more first arms includes a plurality of openings, the openings configured to releasably couple to the one or more pegs.

In accordance with an embodiment of the present invention, the one or more first arms couple with the proximal end of the catheter adapter.

In accordance with an embodiment of the present invention, the one or more first arms couple with the distal end of the catheter adapter.

In accordance with an embodiment of the present invention, the one or more first arms couple with the catheter adapted at a plurality of locations.

In accordance with an embodiment of the present invention, the one or more first arms are formed of a flexible material.

In accordance with an embodiment of the present invention, the one or more first arms are formed of an elastomeric material.

In accordance with an embodiment of the present invention, the one or more first arms are formed of a rigid material.

In accordance with an embodiment of the present invention, the catheter adapter includes one or more second arms integral with the pair of wings, the one or more arms configured to couple with the tourniquet.

Also provided herein is a catheter assembly, including a catheter adapter having a proximal end, a distal end, and a lumen extending along a first longitudinal axis therebetween, a catheter extending from the distal end of the catheter adapter, an extension tube extending from the proximal end of the catheter adapter, and a tourniquet configured to apply a radially-inward pressure to a limb of a patient near the site of penetration, the tourniquet configured to couple with the extension tube at one or more positions along a length of the extension tube.

In accordance with an embodiment of the present invention, a dressing is configured to cover at least a portion of the catheter adapter at a site of penetration into a patient's vasculature, including a proximal end, a distal end, first and second sides arranged between the proximal end and the distal end, the proximal end, distal end, and first and second sides defining a second longitudinal axis and configured to cover at least a portion of the catheter adapter at a site of penetration into a patient's vasculature, an upper surface, a lower surface configured to contact skin of a patient, and a wire configured to couple with the extension tube.

In accordance with an embodiment of the present invention, the catheter adapter includes a pair of wings arranged about the lumen, and wherein the one or more wings are configured to couple with the extension tube.

In accordance with an embodiment of the present invention, the dressing is configured to couple with the extension tube.

In accordance with an embodiment of the present invention, the tourniquet, the wings, and/or the dressing couple with the extension tube via a snap fit.

Also provided herein is a method collecting blood from a patient's vasculature, including steps of attaching the catheter assembly of any of claims 1-40 to a limb of the patient, applying radially-inward compression to the limb with the tourniquet, and attempting to collect blood from the catheter.

In accordance with an embodiment of the present invention, when blood cannot be collected from the catheter, the method includes applying traction to the catheter assembly, and attempting, for a second time, to collect blood from the catheter.

In accordance with an embodiment of the present invention, when blood cannot be collected from the catheter during the second attempt, the method includes applying traction to the catheter assembly with the tourniquet, and attempting, for a third time, to collected blood from the catheter.

### BRIEF DESCRIPTION OF THE DRAWINGS

FIG. 1 is a perspective view of a catheter assembly according to non-limiting embodiments or aspects as described herein;

FIG. 2 is a perspective view of a catheter assembly according to non-limiting embodiments or aspects as described herein;

FIG. 3 is a perspective view of a catheter assembly according to non-limiting embodiments or aspects as described herein;

FIG. 4 is a perspective view of a catheter assembly according to non-limiting embodiments or aspects as described herein;

FIGS. 5A-5C are top and side views a catheter assembly according to non-limiting embodiments or aspects as described herein;

FIGS. 6A-6B are top views of a catheter assembly according to non-limiting embodiments or aspects as described herein;

FIG. 7 is a perspective view of a catheter assembly according to non-limiting embodiments or aspects as described herein;

FIG. 8 is a top view of a catheter assembly according to non-limiting embodiments or aspects as described herein;

FIG. 9 is a top view of a catheter assembly according to non-limiting embodiments or aspects as described herein;

FIG. 10 is a top view of a catheter assembly according to non-limiting embodiments or aspects as described herein;

FIGS. 11A and 11B are top and side views of a catheter assembly according to non-limiting embodiments or aspects as described herein;

FIG. 12 is a top view of a catheter assembly according to non-limiting embodiments or aspects as described herein;

FIG. 13 is a top view of a catheter assembly according to non-limiting embodiments or aspects as described herein;

FIG. 14 is a perspective view of a catheter assembly according to non-limiting embodiments or aspects as described herein;

FIG. 15 is a perspective view of a catheter assembly according to non-limiting embodiments or aspects as described herein; and

FIG. 16 is a top view of a catheter assembly according to non-limiting embodiments or aspects as described herein.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

It should be understood that any numerical range recited herein is intended to include all values and sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

Provided herein is a catheter assembly that allows a user to apply traction to an indwelling IV catheter to free the tip thereof from occlusions that could prevent withdrawing blood from, or injecting a composition through, the catheter. The traction may be applied in a temporally commensurate manner with application of a tourniquet, which can also improve withdrawal of blood through the catheter.

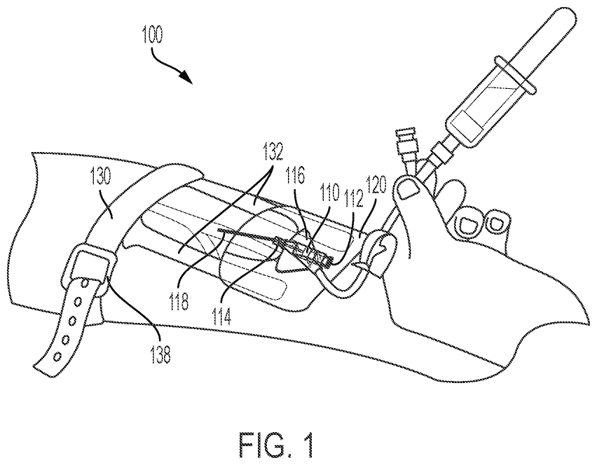

Referring to FIG. 1, shown is a non-limiting embodiment of a catheter assembly 100, which may include a catheter adapter 110 having a proximal end 112, a distal end 114, and a lumen (not shown) therebetween, fluidly connecting the proximal end 112 and the distal end 114. Catheter adapter proximal 112 and distal ends 114 can define a first longitudinal axis. Catheter adapter 110 may include, at distal end 114 thereof, a catheter 118, configured to be arranged within a patient's vasculature and in fluid communication with the lumen. Catheter adapters 110 useful in the assemblies described herein can include any number of features known to those of skill in the art, such as luer connections or needleless access connectors at proximal end 112 thereof, and/or one or more sideports providing fluid communication between an extension set and the lumen of the catheter adapter 110, and other like features. Needleless access connectors are known to those of skill in the art and are commercially available from, for example, Becton, Dickinson and Company under the tradenames SMARTSITE and Q-SYTE. Extension sets are known to those of skill in the art and are commercially available from, for example, Becton, Dickinson and Company under the tradenames MAXPLUS, MAXZERO, NEUTRACLEAR, Q-SYTE, and SMARTSITE. Catheter 118 may be a peripheral intravenous catheter, a midline catheter, or a peripherally-inserted central catheter.

With continuing reference to FIG. 1, catheter assembly 100 may include dressing 120 having a first end, a second end, and first and second sides therebetween, defining a second longitudinal axis that may be substantially parallel to the first longitudinal axis of catheter adapter 110. While dressing 120 is exemplified in the attached drawings as being of a substantially square or rectangular shaped, those of skill in the art will appreciate that any shape may be used, so long as dressing 120 can include features as described herein. With reference to the non-limiting embodiment of FIG. 1, the ends and sides of dressing 120 define a perimeter, and, when in place on a patient, dressing 120 covers at least a portion of catheter adapter 110, such that at least a portion of catheter adapter 110 is located within the perimeter. Dressing 120 includes a top surface and a bottom surface, the bottom surface configured to contact the skin of a patient. In non-limiting embodiments, dressing 120 includes an adhesive on the bottom surface, for improved adherence to the skin. Suitable dressings may also include antimicrobial compositions, for increasing resistance to infections that can be common with indwelling catheters.

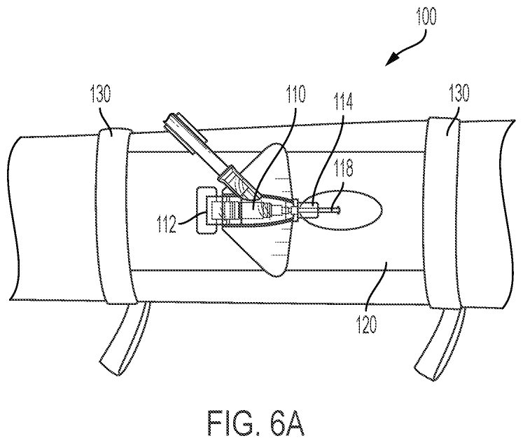

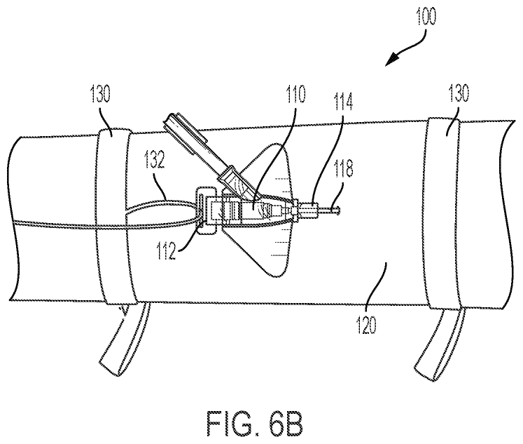

Catheter assembly 110 may further include a tourniquet 130, which may be configured as a removably attachable component of the catheter assembly 110, configured to surround a limb of a patient and substantially or completely occlude flow of blood through one or more vessels thereof. Tourniquet 130 may be formed of any suitable material, such as an elastomeric material, a fabric material, a woven or non-woven material, and/or a film-like material. In non-limiting embodiments, tourniquet 130 is an inflatable device, optionally a reversibly inflatable device, that, when inflated compresses one or more blood vessels below the skin at the site of application, thereby substantially or completely occluding flow of blood therethrough. Tourniquet 130 may include a fastener 138, for example a hook and loop fastener, a pin and hole fastener, a releasable adhesive layer, a wrap and tie, or other like fastener to allow a user to adjust the length of the tourniquet, provide a radially compressive force to the patient, and occlude one or more blood vessels. Without wishing to be bound by the theory, it is believed that applying a tourniquet downstream of catheter 118, occlusion of the vessels causes distension of the vessels as blood collects, making blood withdrawal from the vessel in which catheter 118 is located far easier. Tourniquet 130 may be releasably, or permanently, coupled with dressing 120 and/or catheter adapter 110, as shown in the accompanying figures. In non-limiting embodiments, for example as shown in FIG. 6, a plurality of tourniquets may be included with catheter assembly 100.

With further reference to FIG. 1, tourniquet may include one or more arms 132 extending therefrom. One or more tourniquet arms 132 may be formed of the same material as the tourniquet, or may be a different material. Arms 132 may be flexible, elastomeric, rigid, semi-, rigid, may provide tension and/or a compressive force, may be releasably or permanently coupled with tourniquet 130, may be loaded in terms of providing or preventing axial or rotational displacement, and/or may loaded, or adjustable passively or actively in terms of the amount of force (tension or compression) they provide. In non-limiting embodiments, tourniquet 130 and/or arms 132 are configured to apply tension and/or compression to dressing 120 and/or catheter adapter 110 based on tightening or loosening of tourniquet 130. In non-limiting embodiments, one or more tourniquet arms 132 may be reversibly, or permanently, coupled with dressing 120 and/or catheter adapter 110, as shown in the accompanying figures.

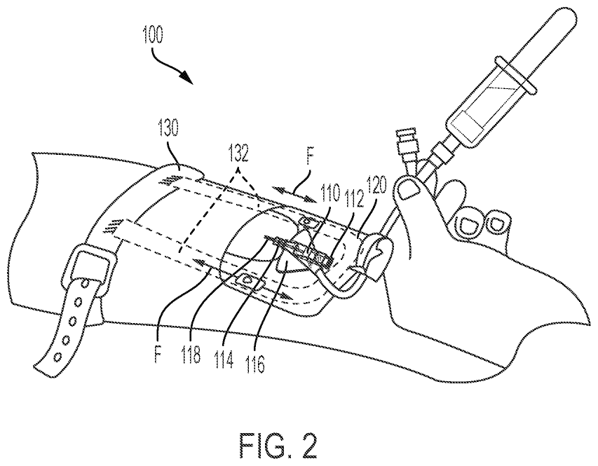

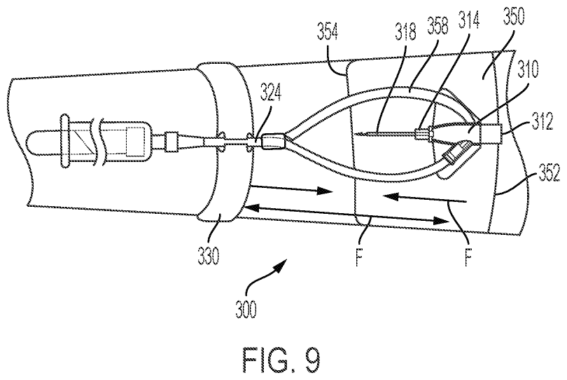

With reference to FIGS. 2-4, one or more tourniquet arms 132 may be reversibly, or permanently, coupled with dressing 120 and/or catheter adapter 110 at one or more connection points 140. In non-limiting embodiments, one or more tourniquet arms are coupled, at one or more locations along their length, to one or more connection points, thereby allowing the amount of tension and/or compression (both indicated as F in FIGS. 2 and 9) to be adjusted and applying traction to catheter 118. As used herein, “traction” means displacement of catheter 118 some distance, either upstream or downstream, from a resting position. In non-limiting embodiments, catheter 118 is displaced in a range of from about 0 to about 0.5 of an inch, all subranges and values therebetween included. In non-limiting embodiments, catheter 118 is displaced about 0.125 of an inch. Without wishing to be bound by the theory, it is believed that by applying force to catheter adapter 110 (e.g., tension and/or compression), by directly coupling tourniquet 130 and/or tourniquet arms 132 with catheter adapter 110 or dressing 130, which may itself be directly or indirectly coupled with catheter adapter 110), a tip of catheter 118 can be displaced, thereby freeing or repositioning the catheter away from potential occlusions (e.g., fibrin sheaths, thrombi, vein walls, and/or valves). While not shown in FIGS. 2-4, but as described and shown later, those of skill in the art will appreciate that catheter adapters can be used with various accessories, such as stabilization devices, devices that serve to adjust the angle at which a catheter adapter rests relative to a patient's skin, and/or anchor pads, for example, those commercially available from Becton, Dickinson and Company under the tradename STATLOCK. Those of skill will appreciate that tourniquets and tourniquet arms as described herein can be coupled with such devices, in a similar manner to coupling with catheter adapters and dressings as described herein, to apply tension and/or compression, and thus to apply traction to a catheter.

In non-limiting embodiments, one or more connection points 140 are releasable connection points for tourniquet 130 and/or arms 132, such as hook and loop connections, adhesive connections, and the like. In non-limiting embodiments, one or more connection points 140 are pegs, extending radially (relative to the longitudinal axis of catheter adapter 110) outward, away from the patient's skin. Connection points 140, such as pegs, maybe integral to dressing 130 and/or catheter adapter 110. In non-limiting embodiments, connection points 140 may be provided as a separate element, releasably or permanently attached to dressing 130, secured thereto by an adhesive, weld, loop and hook fastener, stitch, or other suitable attachment. Connection points 140 can include any features suitable to prevent tourniquet 130 and/or arms 132 from inadvertently decoupling from connection points 140 during application of tension or compression to dressing 120 and/or catheter adapter 110.

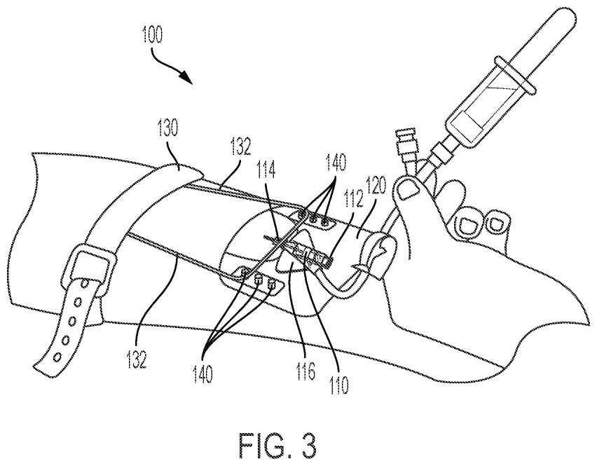

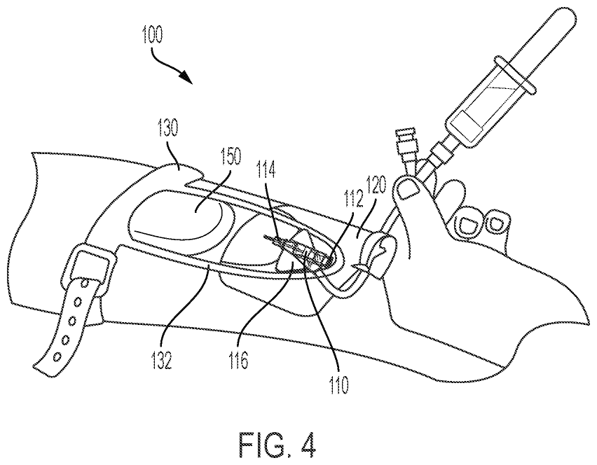

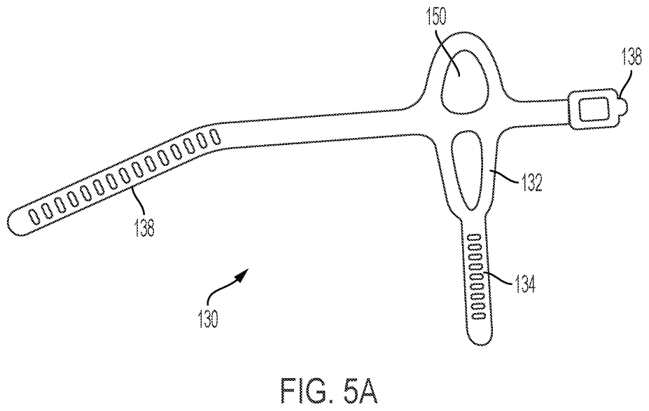

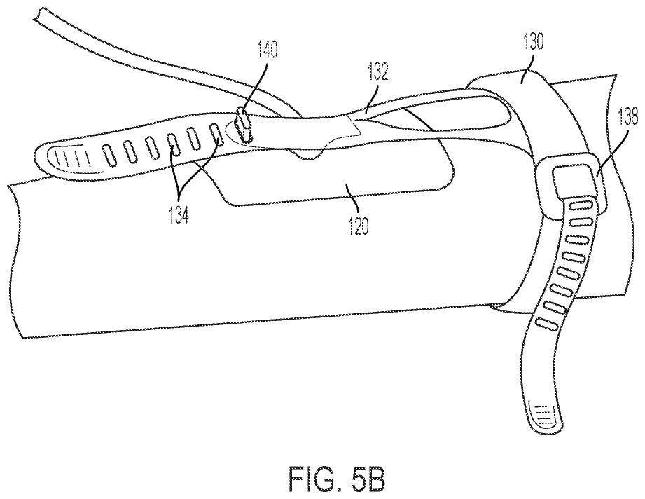

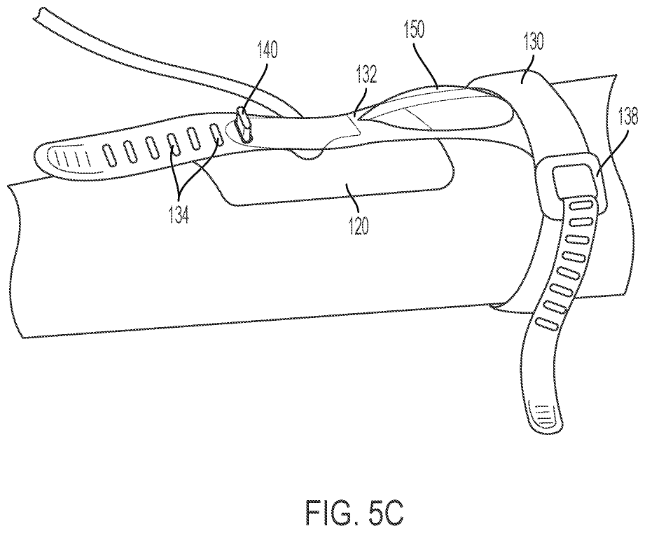

In non-limiting embodiments, for example as shown in FIGS. 5A-5C, one or more tourniquet arms 132 may include one or more openings 134 therein, for coupling with, by receiving, one or more connection points 140. Connection points may be arranged in any suitable configuration to provide tension or compression to dressing 120 and/or catheter adapter, thereby applying traction to catheter 118. For example, connection points 140 may be arranged at one or more locations along one, or both, sides of dressing 120, along the second longitudinal axis, as shown for example in FIG. 3. In non-limiting embodiments, one or more connection points 140 may be arranged along one, or both, ends and/or sides of dressing 120. In non-limiting embodiments, one or more connection points 140 may be arranged on catheter adapter 110, for example on proximal end 112, distal end 114, and/or wings 116. For example, as shown in FIG. 4, one or more arms 132 couple with connection point 140 arranged at proximal end 112 of catheter adapter 110. In the embodiment illustrated in FIG. 4, one or more arms 132 are arranged as a loop that couples with connection point 140, providing equal tension or compression along both sides of catheter adapter 110, reducing rotational displacement of catheter 118.

Turning to FIG. 4, in non-limiting embodiments, catheter assembly 100 may include an enclosure 150 configured cover at least a portion of dressing 120 and/or catheter adapter 110. In non-limiting embodiments, enclosure 150 is a vacuum dome configured to allow for the application of vacuum, for example through a vacuum pump, at or adjacent to the site of catheterization, thereby generating a negative pressure environment under the dome and improving blood withdrawal through catheter 118.

Turning to FIGS. 5A-5C, shown is a non-limiting embodiment of a tourniquet 130 and arm 132 for use with a catheter assembly as described herein. In the illustrated embodiment, tourniquet 130 includes a fastener 138, in the form of a pin and hole connection, an arm 132 including a number of openings 134 along the length thereof. The different openings 134 allow, at least in part based on which opening 134 is utilized with the pin connection and the material from which tourniquet 130 and/or arm 132 are made, for variable levels of tension or compression to be applied to a dressing 120 or catheter adapter (as shown in FIGS. 5B and 5C), thereby applying traction to the catheter. Optionally, for example as shown in FIGS. 5A and 5C, tourniquet 130 includes an enclosure 150, such as a vacuum dome.

Turning to FIGS. 6A and 6B, shown are non-limiting embodiments of a catheter assembly 100 including a catheter adapter 110, dressing 120, and a plurality of tourniquets 130, one arranged downstream of catheter 118, and one arranged upstream of catheter 118. Use of a second tourniquet 130 provides several different features to catheter assembly 100. Use of a second tourniquet 130 allows for traction in both directions (e.g., pushing and pulling catheter 118). This can be accomplished without (FIG. 6A) or with (FIG. 6B) one or more arms 132 coupling tourniquets 130 to dressing 120 and/or catheter adapter 110. In non-limiting embodiments, such as illustrated in FIG. 6B, tourniquet 130 and dressing 120 and/or catheter adapter 110 can be coupled such that tightening tourniquet 130 in preparing for blood withdrawal can simultaneously apply traction to catheter 118.

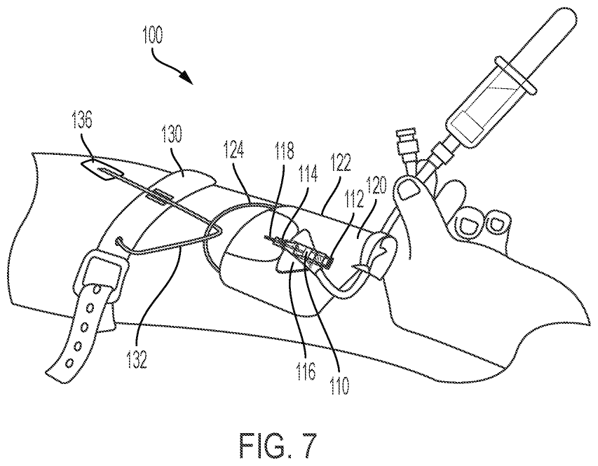

Turning to FIG. 7, shown is a non-limiting embodiment of a catheter assembly 100, including a catheter adapter 110, dressing 120, and tourniquet 130. As described previously, catheter adapter 110 may have proximal end 112 and distal end 114, with a catheter 118 extending from distal end 114. As described previously, tourniquet 130 may include tourniquet arm 132. In the non-limiting embodiment shown in FIG. 7, tourniquet arm 132 is coupled with tourniquet 130 at a plurality of locations. While FIG. 7 shows tourniquet arm 132 coupled with dressing 120 at a single location at distal end 124 thereof, it is to be appreciated that tourniquet arm 132 may be coupled with dressing 120 and/or catheter adapter 110 at any suitable locations. In the non-limiting embodiment of FIG. 7, tourniquet arm 132 can be tensioned with tensioning assembly 136, providing traction to catheter 118 through the coupling of tourniquet arm to dressing 120 and/or catheter adapter 110.

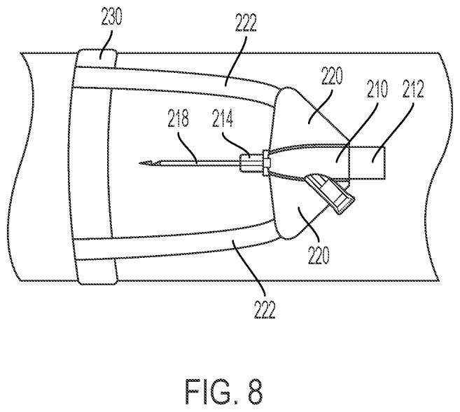

Turning to FIG. 8, shown is a non-limiting embodiment of a catheter assembly 200 including a catheter adapter 210 having a first end 212 and second end 214 defining a lumen (not shown) therebetween, and a catheter 218 at the second end 214. Catheter assembly 200 may include a tourniquet 230. In the non-limiting embodiment shown in FIG. 8, catheter adapter 210 includes one or more wings 220 and one or more arms 222 extending therefrom. One or more catheter adapter arms 222 may be reversibly coupled, or integral to, one or more wings 220, and may couple with tourniquet 230 and/or tourniquet arms (not shown). In non-limiting embodiments, one or more catheter adapter arms 222 may be formed of the same material as, or a different material than, catheter adapter 210. In non-limiting embodiments, one or more catheter adapter arms 222 may be overmolded as part of catheter adapter 210, for example as part of one or more catheter adapter wings 220. As described above, coupling of catheter adapter 210, here through arms 222, with tourniquet 230 or tourniquet arms, allows for application of tension and/or compression to catheter adapter 210, and, thus, traction to catheter 218.

Turning to FIG. 9, shown is a non-limiting embodiment of a catheter assembly 300 including a catheter adapter 310 having a proximal end 312 and distal end 314 defining a longitudinal axis, and including a lumen (not shown) therebetween, and a catheter 318 at the distal end. Catheter assembly 300 may include tourniquet 330 and dressing 350 having a first end 352 and a second end 354. In the non-limiting embodiment of FIG. 9, catheter assembly 300 includes an extension set, including extension tube 324 in fluid communication with lumen (not shown) of catheter adapter 310. As shown in FIG. 9, extension tube 324 may be utilized to provide traction to catheter 318, by coupling with tourniquet 330. In this way, by applying tension to extension tube 324, for example by pulling extension tube towards tourniquet 330 and coupling extension tube 324 with tourniquet 330, varying amounts of traction can be applied to catheter 318. In non-limiting embodiments a second extension tube (not shown) may be utilized, attached to a second tether and/or tourniquet, to apply traction in a plurality of directions. Extension tube 324 may be coupled with tourniquet 330 through any suitable type of connection, for example through a snap fit, friction fit, or the like. In non-limiting embodiments, catheter assembly 300 may include an additional tether 358. Tether 358 can be formed of any useful material, such as those disclosed above as suitable for tourniquet arms or catheter adapter arms. Tether 358 may be coupled with dressing 350 and/or catheter adapter 310, as described previously.

In non-limiting embodiments, tether 358 is coupled with extension tube 324. In non-limiting embodiments, tether 358 is coupled at one end thereof with extension tube 324, and is coupled at another end thereof with catheter adapter 310. Tether 358 may be coupled with catheter adapter 310 at a location opposite that of extension tube 324, such that the combination of tether 358 and extension tube 324 provides substantially equal tension and/or compression to catheter adapter 310 when extension tube 324 is coupled with tourniquet, thereby providing traction to catheter 318 without any significant rotational force, for example about the longitudinal axis of catheter adapter 310, being applied to catheter 318. In non-limiting embodiments, tether 358 is at least partially embedded in dressing 350.

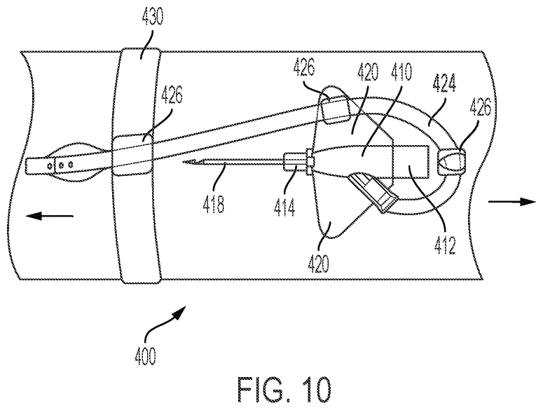

Turning to FIG. 10, shown is a non-limiting embodiment of a catheter assembly 400 including a catheter adapter 410 having a proximal end 412 and distal end 414 defining a longitudinal axis, and including a lumen (not shown) therebetween, and a catheter 418 at the distal end. Catheter adapter 410 may further include one or more wings 420. One or more wings 420 may be arranged in any suitable manner, for example extending between proximal end 412 and distal end 414 of catheter adapter 410. In the non-limiting embodiment of FIG. 10, catheter assembly 400 includes an extension set, including extension tube 424 in fluid communication with lumen (not shown) of catheter adapter 410. Similar to the embodiment shown in FIG. 9, in the embodiment of FIG. 10 extension tube 424 may be utilized to provide traction to catheter 418, by coupling with tourniquet 430. In this way, by applying tension to extension tube 424, for example by pulling extension tube towards tourniquet 430 and coupling extension tube 424 with tourniquet 430, varying amounts of traction can be applied to catheter 418. Extension tube 424 may be coupled, reversibly or permanently, with one or more of tourniquet 430, catheter adapter 410, and/or a dressing (not shown) at one or more connectors 426, through any suitable type of connection, for example through a snap fit, friction fit, or the like. In non-limiting embodiments, extension tube 424 may couple both with catheter adapter 410 and tourniquet 430 at connectors 426. For example, as shown in FIG. 10, extension tube 424 may couple with one or more wings 420 of catheter adapter 410. Extension tube 424 may be coupled with catheter adapter, for example with one or more wings 420 thereof, through any suitable type of connection, for example through a snap fit, friction fit, or the like. In non-limiting embodiments, extension tube 424 may couple a wing 420 of catheter adapter 410 on a side opposite a side of the catheter adapter 410 where an end of extension tube 424 is coupled for being in fluid communication with lumen. In this way, extension tube 424 may, when tensioned through its coupling with tourniquet 430, provide substantially equal tension and/or compression to catheter adapter 410, thereby providing traction to catheter 418 without any significant rotational force, for example about the longitudinal axis of catheter adapter 410, being applied to catheter adapter 410.

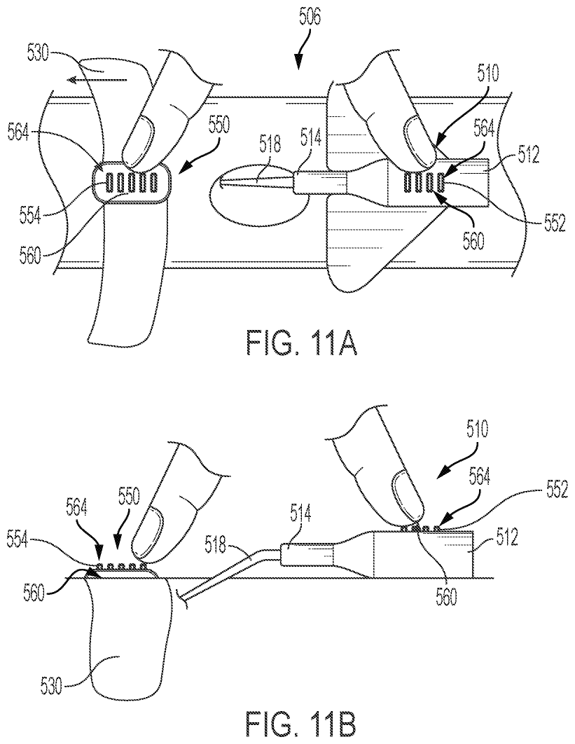

Turning to FIGS. 11-16, shown are non-limiting embodiments of a catheter assembly 500, including devices for providing traction to a catheter 518, with one or more added tourniquets 530. Such traction devices are disclosed in International Patent Application Publication No. PCT/US2021/36859, the content of which is incorporated herein by reference in its entirety.

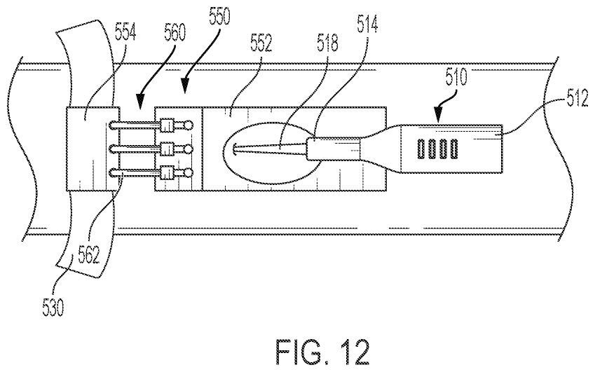

With regard to FIGS. 11A-12, shown are non-limiting embodiments of a catheter assembly 500 configured to apply traction to a catheter 518 to create a clear pathway for fluid flow. In some embodiments, the catheter assembly 500 may include a catheter adapter 510 having a proximal end 512, a distal end 514, and a lumen (not shown) extending along a longitudinal axis therebetween.

The catheter 518 may extend from the distal end 514 of the catheter adapter 510. In some embodiments, the catheter 518 may be used for blood collection, fluid delivery, patient or device monitoring, or other clinical needs. In some embodiments, the catheter 518 may include, for example, a peripheral IV catheter, a peripherally-inserted central catheter, or a midline catheter. In some embodiments, the catheter 518 may have been previously inserted into the vasculature of the patient and may be dwelling within the vasculature.

In some embodiments, a dressing 550 may securely apply traction to the catheter 112 while the catheter 112 is disposed within the vasculature of the patient. In some embodiments, the dressing 550 may include an adhesive 126 to secure the dressing 550 to the patient. In some embodiments, the dressing 550 may include a two-piece or two-section dressing 550 connected by an adjustment element 560. In some embodiments, the dressing 550 may include more than two sections, and may be connected by one or more adjustment elements 560, such as an adjustable tether or other suitable fastener or strap.

In some embodiments, each or some of the sections of the dressing 550 may be spaced apart from each other by a distance. The distance between the sections of the dressing 550 may be selected to optimize application of traction to the catheter 518. For example, in operation, some embodiments may apply traction to the catheter 518 by adjusting the distance between two or more of the dressing 550 pieces or sections. In some embodiments, the distance between the dressing 550 sections may be manually or automatically adjusted by the adjustment element 560. In some embodiments, adjusting the distance between the dressing 550 sections in this manner may exert tension between the dressing 550 sections to apply traction to the catheter 518.

In some embodiments, the dressing 550 may include a first section 552 adjustably coupled with a second section 554. In some embodiments, an adjustment element 560 may adjustably couple the first section 552 to the second section 554. The adjustment element 560 may include, for example, a tether, a winch, a ratchet, a pulley, a dial, a screw, a cable tie, or other suitable tensioning device or mechanism. Some embodiments of the adjustment element 560 may be releasable and/or removable to decouple the first and second sections 116, 118.

In some embodiments, the first section 552 may be independent of the second section 554. Some embodiments of the first section 552 may be configured to secure and/or maintain a position of the catheter adapter 102 and/or the catheter 518 to the patient via an adhesive 126, for example. In some embodiments, the first section 552 may include an aperture 124 or other suitable feature or mechanism to maintain the catheter adapter 102 and/or the catheter 518 in a substantially fixed position relative to the patient.

In some embodiments, the second section 554 may be adjustably positioned and/or secured relative to the first section 552 to apply traction to the catheter 518 within the vasculature. In some embodiments, the second section 554 may include an adhesive or other suitable device or mechanism to secure and/or maintain the position of the second section 554 relative to the patient. Some embodiments of the second section 554 may include a securing device or mechanism to secure and/or maintain the position of the second section 554 relative to the first section 552.

In some embodiments, the position of the second section 554 relative to the first section 552 may be adjusted by the adjustment element 560. In some embodiments, as shown in FIG. 1, the adjustment element 560 may include one or more tethers 562 to couple the dressing 550 sections 552, 554 together. In some embodiments, a first end of the tether 562 may be coupled with the first section 552 and a second end of the tether 562 may be coupled with the second section 554.

In some embodiments, the tethers 562 may be configured to be adjusted individually such that each tether 562 individually pulls traction on the catheter 518. In some embodiments, the tethers 562 may be configured to be adjusted collectively as a single unit to pull traction on the catheter 518. In some embodiments, each tether 562 may pull traction on the catheter 518 in either a proximal or distal direction.

In some embodiments, tightening the tethers 562 may pull the skin of the patient such that the tip of the catheter 518 may be re-oriented within the vasculature. In some embodiments, repositioning the tip of the catheter 518 within the vasculature in this manner may clear a fluid path for infusion of fluid or medications or blood withdrawal. In some embodiments, the adjustment element 560 may be automatically or manually adjusted or loosened to return the dressing 550 to its initial position, thereby releasing traction on the catheter 518.

As shown in FIGS. 11A-12, catheter assembly 500 may include one or more tourniquets 530 coupled with traction device 560 and/or dressing 550, as described previously. The combination of one or more tourniquets 530 and traction device 560 may improve blood withdrawal or infusion of medications, for the reasons described previously.

In some embodiments, the adjustment element 560 may include one or more finger grips 564 to facilitate manual adjustment of the second section 554 of the dressing 550 relative to the first section 552 of the dressing 550. In some embodiments, a first finger grip 564 may be coupled with the first section 552 and a second finger grip 564 may be coupled with the second section 554. In some embodiments, the first section 552 and the second section 554 of the dressing 550 may be monolithically formed as a single unit. Some embodiments of finger grips 564 may be coupled with the catheter adapter 510.

In operation, a user, such as a medical professional, may utilize the finger grips 564 to adjust or maintain the position of the first section 552 relative to the patient while utilizing another finger grip 564 to adjust or maintain the position of the second section 554 relative to the first section 552. In some embodiments, the finger grips 564 may be utilized to maintain the position of the first section 552 while the position of the second section 554 may be adjusted relative to the first section 552 via another finger grip 564.

In some embodiments, a finger grip 564 may be utilized to adjust the position of the first section 552 in a proximal or distal direction while the position of the second section 554 may be simultaneously adjusted in an opposite direction via another finger grip 564. In some embodiments, the finger grips 564 may be utilized to adjust a tilt, incline, or other position or orientation of the first section 552 relative to the second section 554. In some embodiments, utilizing one or more finger grips 564 to manually adjust the position of the first section 552 relative to the second section 554 in this manner may apply traction to the catheter 518 to reposition the tip of the catheter 518 within the vasculature and thereby open a fluid path.

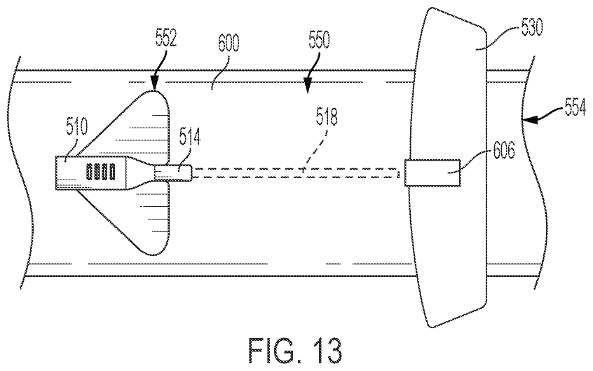

Referring now to FIG. 13, in some embodiments, catheter assembly 500 may include a dressing 550 where the first section 562 includes a pad 600 to secure the catheter adapter 510 relative to the patient. The pad 600 may include an aperture, adhesive, and/or other suitable securing feature to receive the catheter adapter 510 and to secure the catheter adapter 510 relative to the pad 600 and the patient. In some embodiments, the catheter 518 may extend from the distal end 514 of the catheter adapter 510, through the aperture or other pad 600 feature, and into the vasculature of the patient. In some embodiments, the pad 600 may have a length sufficient to extend over a length of the catheter 518 within the vasculature. In some embodiments, the pad 600 may include markings to indicate an approximate location of the catheter 518 beneath the pad 600.

In some embodiments, the second section 554 may include an isolated section of the dressing 550, such as a tab 606 coupled with a distal end of the pad 600. In some embodiments, the pad 600 and the tab 606 may be monolithically formed as a single unit. In operation, the tab 606 may be pulled in a distal direction relative to the distal end 514 of the catheter adapter 510 to apply traction to the catheter 518. In some embodiments, the tab 606 may include an adhesive or other suitable mechanism to enable the tab 606 to be selectively moved in the distal direction and secured in place to apply continuous traction to the catheter 518. In some embodiments, the markings and/or the tab 606 may facilitate blood withdrawal by indicating a desired or optimal location for a clinician to apply pressure for blood withdrawal.

As shown in FIG. 13, catheter assembly 500 may include one or more tourniquets 530 coupled with tab 606 of dressing 550, as described previously. The combination of one or more tourniquets 530 and tab 606 may improve blood withdrawal or infusion of medications, for the reasons described previously.

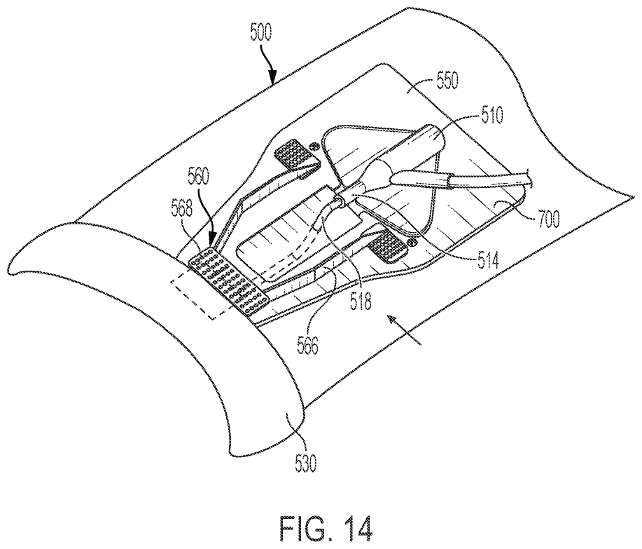

Referring now to FIG. 14, in some embodiments, the catheter assembly 500 may include the dressing 550 to receive the catheter adapter 510 and a traction mechanism 560 coupled with the dressing 550 to consistently apply traction to the catheter 518 extending from the catheter adapter 510. In some embodiments, the dressing 550 may include a stabilization pad 700 having a contour to receive and stabilize the catheter adapter 510 and/or a stabilizer element coupled thereto. Suitable stabilization pads are commercially available from, for example, Becton, Dickinson and Company under the tradename STATLOCK.

Some embodiments of the traction mechanism 560 may be coupled with the stabilization pad 700 at a location substantially corresponding to a position of the catheter 518. In some embodiments, the traction mechanism 560 may include anchor points located on either side of the catheter 518 at or near its proximal end. In some embodiments, traction arms 566 may extend distally from the anchor points parallel to the catheter 518. In some embodiments, a traction plate 568 extending in a substantially transverse direction relative to the traction arms 566 may be coupled with or integrated with the distal ends of the traction arms 566. In some embodiments, the traction plate 568 may provide a distal anchor point for the traction mechanism 560.

In operation, in some embodiments, the traction arms 566 may be squeezed inwardly to straighten or to deform a contour of each of the traction arms 566 in an inward direction relative to the catheter 518. In some embodiments, this application of force may apply controlled traction to the catheter 518 to open a fluid path within the vasculature.

As shown in FIG. 14, catheter assembly 500 may include one or more tourniquets 530 coupled with traction plate 568. The combination of one or more tourniquets 530 and traction plate 568 may improve blood withdrawal or infusion of medications, for the reasons described previously.

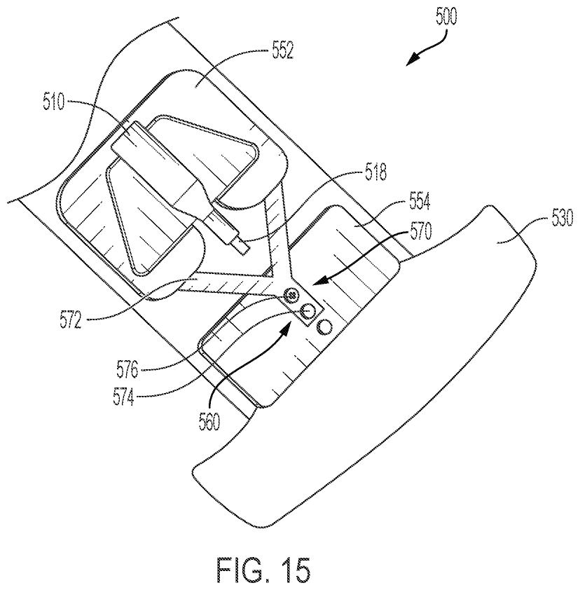

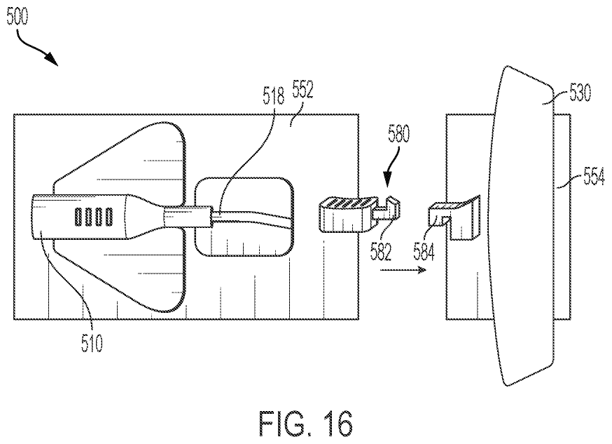

Referring now to FIGS. 15 and 16, in some embodiments, the first section 552 of the dressing 550 may include a contour, adhesive and/or other feature to retain the catheter adapter 510 in a substantially fixed position relative to the patient. The second section 554 of the dressing 550 may be spaced apart from the first section 552 and may be substantially fixed relative to the patient via an adhesive or other suitable device or mechanism. In this manner, in some embodiments, a substantially constant distance may separate the first section 552 and the second section 554.

In some embodiments, the traction mechanism 560 may be coupled with the first section 552 of the dressing 550 and may be configured to attach to the second section 554 of the dressing 114 to adjust the distance between the same. In some embodiments, the traction mechanism 560 may include an adjustable snap feature 570 to adjust the distance between the first section 552 and the second section 554. In this manner, some embodiments of the snap feature 570 may apply consistent traction to the catheter 518.

As shown in FIG. 15, in some embodiments, the traction mechanism 560 may include a tension strap 572 coupled with the first section 552 of the dressing 550. In some embodiments, the tension strap 572 may be monolithically formed as a single unit with the first section 116 of the dressing 550. In some embodiments, the catheter adapter 510 and/or stabilizer element may be coupled with the tension strap 572. In other embodiments, the tension strap 572 may substantially surround the catheter adapter 510 and/or stabilizer element.

In any case, some embodiments of the tension strap 572 may include the snap feature 570 coupled with or integrated with its distal end. In some embodiments, the snap feature 570 may include an extension or tab having one or more snap caps 1206 disposed along a length thereof. In some embodiments, each snap cap 574 may be configured to couple to a socket 576 coupled with or integrated with the second section 554 of the dressing 550. In some embodiments, the second section 554 may include multiple sockets 576 such that one or more of the snap caps 574 may be adjustably coupled with one or more of the sockets 576. In this manner, in some embodiments, the tension strap 572 may be tightened and/or loosened relative to the second section 554 of the dressing 550 to adjust traction applied to the catheter 518 in a controlled manner.

As shown in FIG. 15, catheter assembly 500 may include one or more tourniquets 530 coupled with dressing 550, such as second section 554. The combination of one or more tourniquets 530 and dressing 550 may improve blood withdrawal or infusion of medications, for the reasons described previously.

Referring now to FIG. 16, in some embodiments, the tension strap 572 or dressing 550 may include an alternative connection feature 580 such as a clasp, buttons, hook and loop fasteners such as hook and loop, adhesive, or other suitable securement device or feature. As shown, in some embodiments, the connection feature 580 may include a first connector element 582 coupled with the first section 552 of the dressing 550 and a second connector element 584 coupled with the second section 554 of the dressing 550. In some embodiments, the first connector element 582 and/or the second connector element 584 may be adjustable to adjust the distance between the first section 552 of the dressing 550 and the second section 554 of the dressing 550. In some embodiments, the first connector element 582 and the second connector element 584 may interlock or may be otherwise coupled together to reduce the distance between the first section 552 and the second section 554 to thereby apply traction to the catheter 510 in a consistent manner.

As shown in FIG. 16, catheter assembly 500 may include one or more tourniquets 530 coupled with dressing 550, such as second section 554. The combination of one or more tourniquets 530 and dressing 550 may improve blood withdrawal or infusion of medications, for the reasons described previously.

Also provided herein are methods of withdrawing blood from a vascular access device, or remediating a failed, or mitigating a potentially failed, vascular access device. As noted above, providing traction to a vascular access device, such as an indwelling catheter can improve blood withdrawal and/or delivery of medicaments through the catheter. Accordingly, in non-limiting embodiments, a method of withdrawing blood includes providing a catheter assembly as described herein. A user, such as a medical professional, may tighten the tourniquet, thereby occluding one or more of the underlying vessels and causing blood to pool near a tip of the indwelling catheter. At this point, blood withdrawal can be attempted. If blood withdrawal is unsuccessful, traction may be applied to the catheter, for example manually by the user by applying force with a finger. Following the application of traction, a second attempt to withdraw blood can be made. If the second attempt is unsuccessful, further traction may be applied to the catheter, for example by attaching a tourniquet arm, catheter arms, and/or increasing/decreasing tension or compression applied to a tourniquet arm or catheter arm, or between tourniquet and dressing, catheter adapter, connection feature, and/or traction mechanism, as described herein. This step may be repeated, as necessary, until blood can be withdrawn. Following successful withdrawal, the tourniquet may then be loosened or removed.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A catheter assembly, comprising:
a catheter adapter comprising a proximal end, a distal end, and a lumen extending along a first longitudinal axis therebetween;
a catheter extending from the distal end of the catheter adapter;
a dressing comprising a first end, a second end, and first and second sides extending therebetween, the dressing defining a second longitudinal axis and configured to cover at least a portion of the catheter adapter at a site of penetration into a patient's vasculature, the dressing further comprising an upper surface and a lower surface, the lower surface configured to contact skin of a patient; and
a tourniquet configured to apply a radially-inward pressure to a limb of a patient near the site of penetration, the tourniquet configured to couple with the dressing.

2. The catheter assembly of claim 1, wherein the tourniquet comprises one or more arms configured to couple with the dressing.

3. The catheter assembly of claim 2, wherein the one or more arms are configured to be coupled with the dressing at one or more locations along the arms.

4. The catheter assembly of claim 1, wherein the dressing further comprises one or more pegs arranged on the upper surface.

5. The catheter assembly of claim 4, wherein the one or more pegs are arranged along the second longitudinal axis of the dressing, the second longitudinal axis being parallel to the first longitudinal axis.

6. The catheter assembly of claim 4, wherein the one or more pegs comprise at least one peg arranged along the first side and at least one peg arranged along the second side.

7. The catheter assembly of claim 4, wherein the one or more arms are configured to releasably couple to the one or more pegs.

8. The catheter assembly of claim 4, wherein the one or more arms comprise a plurality of openings, the openings configured to releasably couple to the one or more pegs.

9. The catheter assembly of claim 2, wherein the one or more arms are connected to the tourniquet at a plurality of locations.

10. The catheter assembly of claim 2, wherein the one or more arms are coupled with the dressing at a single location.

11. The catheter assembly of claim 2, wherein the one or more arms comprise a tensioning assembly.

12. The catheter assembly of claim 2, wherein the one or more arms couple to the dressing adjacent the proximal end of the catheter adapter.

13. The catheter assembly of claim 2, wherein the one or more arms couple to the dressing adjacent the distal end of the catheter adapter.

14. The catheter assembly of claim 2, wherein the one or more arms are formed of a flexible material.

15. The catheter assembly of claim 2, wherein the one or more arms are formed of an elastomeric material.

16. The catheter assembly of claim 2, wherein the one or more arms are formed of a rigid material.

17. The catheter assembly of claim 1, wherein the dressing comprises an adhesive arranged on the lower surface.

18. The catheter assembly of claim 1, further comprising an enclosure configured to cover at least a portion of the catheter adapter.

19. The catheter assembly of claim 1, wherein the tourniquet comprises a fastener configured to allow the tourniquet to be shortened and lengthened, thereby allowing differing amounts of radially-inward pressure to be applied to the patient's limb.

20. The catheter assembly of claim 1, further comprising a second tourniquet configured to apply a radially-inward pressure to a limb of a patient near the site of penetration, the second tourniquet configured to couple with the dressing.

21. The catheter assembly of claim 20, wherein the second tourniquet comprises one or more second arms configured to couple with the dressing.
